(19) United States
(12) Patent Application Publication
(10) Pub. No.: US 2012/0243699 A1
(43) Pub. Date: Sep. 27, 2012

(54) EAR CANAL TRANSDUCER MOUNTING SYSTEM

(76) Inventor: Kevin Michael, Pennsylvania Furnace, PA (US)

(21) Appl. No.: 13/071,484

(22) Filed: Mar. 24, 2011

(51) Int. Cl. A61F 11/06; A61B 1/227; H04R 1/10

(52) U.S. Cl. 381/72; 381/380; 600/559

(57) ABSTRACT

An ear-canal mounted sound transducer system comprises a miniature sound transducer mounted in a soft, acoustically transparent material, and is suitable for shallow, semi-deep or deep placement inside the ear canal of a user, and for use with or without a personal hearing protective device. The sound transducer may be a microphone or a speaker, and is optionally attached to a flat flexible cable, connected to an analyzer or a signal source. Also provided is an improved method for measuring level of noise attenuation using the ear-canal mounted sound microphone.

### FIELD

The present invention relates to an ear canal transducer mounting system, wherein either a microphone or a speaker is mounted inside a user's ear canal, and further to methods of using the system for e.g. measuring noise level attenuation provided by hearing protectors.

### BACKGROUND OF INVENTION

Under many circumstances, there is a need to either measure or deliver sound inside a user's ear canal. For example, a microphone needs to be placed inside an ear canal to measure the user's noise exposure. Similarly, a mini-speaker may need to be placed inside a user's ear canal, e.g. when the user is in a very noisy environment, having to wear a noise protection device yet at the same time in need of voice-radio communication with others. In addition, it may be desirable in general to deliver sound (e.g. music) to the user directly to a location inside a user's ear canal, which when used with conventional hearing protection will avoid noise from the ambient, maximize signal to noise ratio, and at the same time allow the power or energy consumption to be minimized. In this situation, if earplugs are worn the transducer will be required to be placed deeper into the ear canal than if earmuffs are worn.

There are many existing systems that measure sound in, or deliver sound to, the ear canal. Typically, these systems utilize a transducer that is located near the entrance of the ear canal (e.g. ear buds) or part way down the ear canal (e.g. earphone speakers attached to conventional insert type hearing protectors), and are often called ear-occluding transducer mounting systems. These transducers are conventionally mounted in an ear-occluding device that serves to attenuate noise and to hold the transducer in place. For many applications these systems are satisfactory, but they have some disadvantages. For example, the ear occluding device may not fit the wearer well due to ear canal size or shape variations, and therefore the device may not provide sufficient attenuation of noise from the environment, compromising sound reception or transmission. Custom molded or specialized foam plugs with sound channels leading to the transducer can be used as alternatives. While custom-molded products or foam products with sound channels offer significant improvement in fitting and effectiveness, they are expensive to use on a daily basis.

On the other hand, there are situations where the “situational awareness” of the user of an ear canal-mounted communication device must not comprised in any way, for example for many military and police operations. For this purpose, the conventional ear buds or ear-occluding transducers are inadequate because they at least partially occlude the sound from the environment.

The present invention addresses these issues. The sound transducer system of the present invention is mounted in the ear canal, using acoustically transparent padding materials. The user's preferred hearing protector may be used independently to attenuate ambient noise if needed; yet the padding material itself causes no or little impediment of the ambient sound.

### SUMMARY OF THE INVENTION

In one embodiment, the present invention provides an ear-canal mounted sound transducer system. The sound transducer may be a microphone or a speaker. The system of the present invention comprises a miniature sound transducer mounted in a soft, acoustically transparent material, and is suitable for shallow, semi-deep or deep placement inside the ear canal of a user. The sound transducer is optionally attached to a flat flexible cable, which may be further attached to a wire.

In another embodiment, the acoustically transparent material is configured to be easily removable by the user for replacement.

In one embodiment, the acoustically transparent material is an open-cell foam, or another fibrous material or other structure that contacts the ear canal walls and holds the transducer in place.

In one embodiment, the sound transducer may be attached to an antenna for wireless transmission.

In another embodiment, the present invention provides a sound insulated sound transduction system. The sound transduction system comprises a hearing protective device, and an ear-canal mounted sound transducer system as described above, wherein the ear-canal mounted sound transducer system comprises a miniature sound transducer mounted in a soft, acoustically transparent material, suitable for placement inside an ear canal of a user, wherein the sound transducer is attached to a flat flexible cable, or an antenna for wireless transmission. The flat flexible cable may be further attached to a more rugged cable, which in turn is connected to a signal source or an analyzer. A suitable hearing protective device may be an ear muff, or an ear plug.

According to one embodiment of the present invention, the sound insulated sound transduction system according uses as acoustically transparent material an open-cell foam, or another fibrous material or other structure that contacts the ear canal walls and holds the transducer in place.

The present invention further provides a method for measuring the level of noise attenuation of a hearing protecting device worn by a user, the method comprising 1) placing in the ear canal of the user an ear dam-mounted microphone of the present invention, 2) administering to the user a first test sound stimulus and a second test sound stimulus, wherein the first test stimulus is administered with the ear open and the second test stimulus is administered with the ears occluded with the hearing protective device; 3) measuring the sound level detected at the microphone to obtain a first measurement without the hearing protective device on, and a second measurements with the hearing protective device on, and 4) determining the level of sound attenuation of the hearing protective device.

In one embodiment, in the above method, the energy level of the first and the second test sound stimuli are the same and the level of sound attenuation is determined by calculating the arithmetic difference between the two measurements. In another embodiment, the energy level of the second test stimulus is higher than the first test stimulus by a fixed amount (X), and the level of sound attenuation is the sum of the arithmetic difference between the two measurements plus (X). Alternatively, the method of testing of the present invention further comprises administering to the user a third test stimulus which is higher in energy level than the first test stimulus by an amount X, and obtaining a third measurement, wherein the level of sound attenuation is the sum of the arithmetic difference between the first and third measurements, plus X.

A plurality of users can be tested simultaneously according to the present invention, and the hearing protection device may be an ear muff, or an ear plug.

### BRIEF DESCRIPTION OF DRAWINGS

FIG. 1 is a top perspective view showing a sound transducer and two ear dams according to the present invention with different sizes according to an embodiment of the invention.

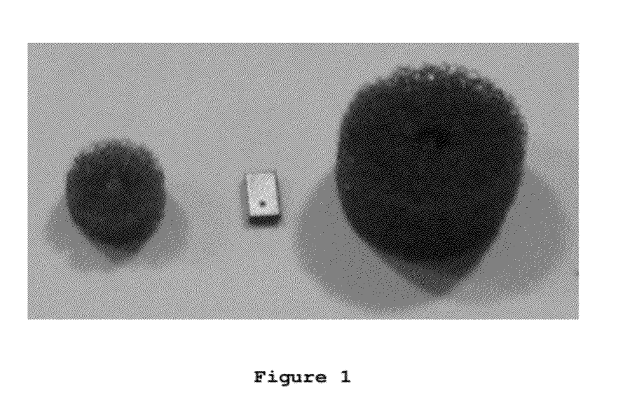

FIG. 2A is a schematic illustration of the placement of a sound transducer into a foam ear dam. The ear dam is in an approximately spherical shape, and a slit or an opening is provided, into which the sound transducer is placed. The side wall of the slit or opening may optionally be provided with an adhesive area on opposite sides for closing. FIG. 2B schematically illustrates the wiring of the sound transducer via a flat flexible cable and connection thereof to a power supply and/or analyzer and/or signal source via a rugged cable.

FIG. 3A is a prior art drawing showing the anatomical structure of the ear canal. FIG. 3B schematically illustrates the placement of a foam-mounted sound transducer of the present invention mounted semi-deeply inside the ear canal.

### DESCRIPTION OF THE INVENTION

The present inventor recognized that by mounting a sound transducer, that is, a microphone or a speaker, inside an open-cell foam or other acoustically transparent padding materials, it becomes feasible to mount the sound transducer inside the user's ear canal. The acoustically transparent padding material contacts the ear canal, which keeps the device stationary and maintains the transducer in a proper position. This ear canal-mounted sound transducer, optionally in combination with a conventional hearing protection device, solves the problems of the prior art outlined above.

The term “open-cell foams”, or structured foams, as used herein, refers to any type of solid foams that contain interconnected pores that form an interconnected network allowing sound transmission. Any open-cell foam that is acoustically transparent is suitable for the present invention. By “acoustically transparent” it is meant that the material allows sound to propagate through it substantially without attenuation. Many acoustically transparent foams or padding materials are commercially available, such as open-celled polyurethane ether or ester foams.

Mounting a piece of open-cell foam into a user's ear canal is known and routinely performed by those skilled in the art. For example, a foam ear dam is routinely used for the manufacture of ear impressions for custom-molded hearing protectors or hearing aid shells. An ear dam is a small piece of open-cell foam, usually shaped as a cube or sphere, but may be in any other suitable shape, offered in various sizes to accommodate different sized ear canals. The process of making ear impressions involves placing the dam deep in the ear canal, then injecting self-curing material into the outer ear area and ear canal. The ear dam functions to block the impression material from flowing too deeply into the ear canal. The material then cures, or hardens, and the ear impression is then gently removed from the ear. The ear dam often has a piece of thread connected to it so that it can be retrieved from the ear canal if it does not stick to the impression material. The impression is then sent to a custom-molded hearing protector manufacturer where it is generally used to make a cast of the ear canal shape, then custom-molded hearing protectors (or hearing aid shells) are manufactured from the cast.

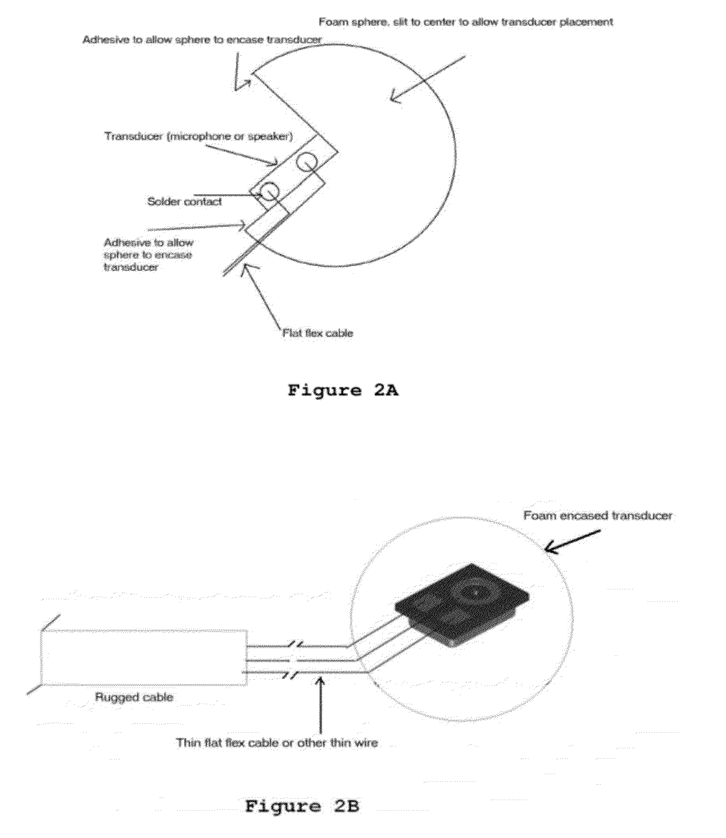

Once formed, the individually fitted ear dams are used for mounting the sound transducer. A schematic illustration of the placement of a sound transducer into a foam ear dam is provided in FIG. 2A. The ear dam may be in an approximately spherical shape, or any other suitable shape as the processing may dictate, and may be cut to provide a slit or an opening. A sound transducer is placed in the opening. The side wall of the slit or opening may optionally be provided with an adhesive area on opposite sides for closing. Alternatively, the foam may be closed via the elasticity of the foam material.

The foam material in which the sound transducer is mounted not only prevents the transducer from touching the sensitive surface of the ear canal or eardrum, but also prevents occlusion of sound transmission due to the transducer being pressed flush against the surface of the ear canal. For example, without the sound-transparent foam, a microphones sound opening may be pressed tightly against the ear canal surface which comprises detection or measurement of the sound.

When mounted, the sound transducer should be completely covered by the foam material. The shape and size of the foam material may vary based on design and production choices, and can be easily determined by those skilled in the art, so long us the user does not feel uncomfortable when the piece is inserted in the ear canal. In addition, the foam is easily removable and replaceable from the transducer for hygiene reasons. The foam may desirably be replaced for each application.

In addition, the transducer needs conductors (e.g. cables or wires) to transmit electrical signals to and from the transducer to a measurement device or signal source, which conductors should only minimally affect the noise attenuation provided by an earplug or earmuff, for example by being thin and as unobtrusive as possible.

A preferred solution for the conductors is a flat flexible cable (FFC) of minimal thickness. Commercially available cables with a thickness of about 2-4 mil (1 mil=0.001 inch) are suitable for this application; and a suitable width for these cables may be approximately 3 mm. A flat flex cable that is about 3 mm wide and about 2-4 mil in thickness can extend under an earplug or earmuff and cause minimal or no loss in noise attenuation. The flat flex cable also provides a mechanism to remove the transducer and encompassing foam from the ear canal.

An alternative solution for transmission of the signal to and from the transducer is a wireless communication system.

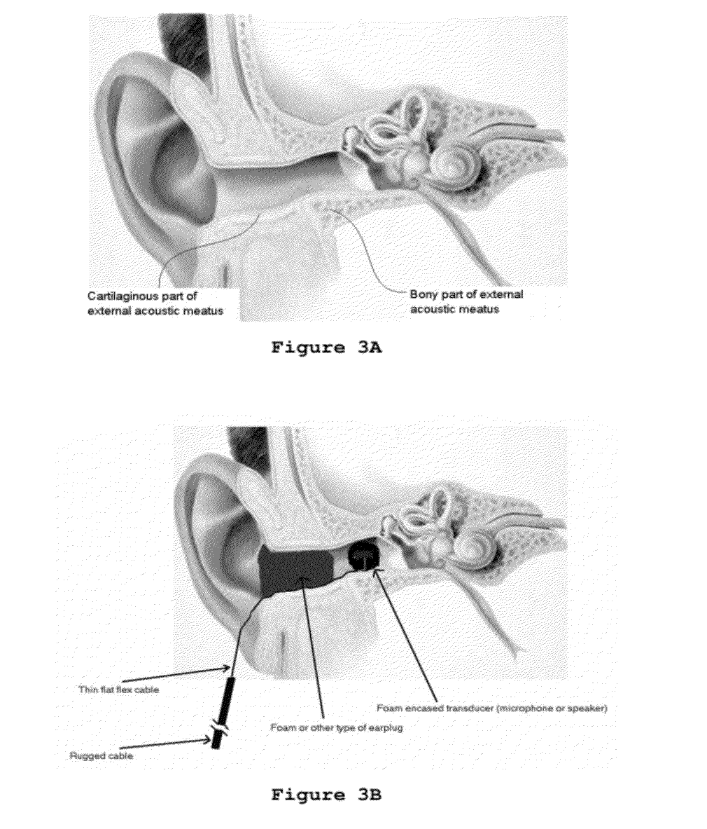

The sound transducer of the present invention may be mounted “deep” or “semi-deep” or “shallow” inside the ear canal of a user. As is well-known, and illustrated in FIG. 3, the adult human ear canal is divided into two parts: the fibrocartilaginous part and the bony part. The fibro-cartilaginous part forms the outer third of the canal, and its anterior and lower wall are cartilaginous, whereas its' superior and back wall are fibrous. The bony part forms the inner two thirds, adjacent to the ear drum. As used in the present invention, “deep” mounting means that foam- or soft material-mounted sound transducer of the present invention is placed completely in the bony part of the ear canal, while by semi-deep mounting, it is meant that the sound transducer is mounted inside the canal in the region that overlaps the fibrocartilaginous and the bony region. Shallow mounting of the transducer means that the transducer mounting structure is placed in the fibro-cartilaginous section of the ear canal.

A sound transducer may be a microphone or a speaker/earphone. A preferred microphone for the present invention is miniature in size. As is readily recognized by those skilled in the art, a sub-miniature microphone or speaker is preferred. Many such sub-miniature microphones and speakers are readily and commercially available.

For example, the Knowles Electronics model SPA2410LR5H-B MEMS type microphone measures 3.3 mm×2.5 mm×1 mm (height). Oriented lengthwise, the cross-sectional dimension of 2.5 mm×1 mm can easily be placed in almost all ear canals.

Similarly, many in-ear loudspeakers or earphones are readily available to those skilled in the art. The Knowles Electronics model FK-23451-000 sub-miniature speaker measures 5.0 mm×2.7 mm×1.9 mm (height). Oriented lengthwise, the cross-sectional dimension of 2.7 mm×1.9 mm can easily be placed in almost all ear canals.

The sound transducer mounted within open-celled foam and placed inside a user's ear canal has many applications. Several non-limiting examples are provided below.

### Determination of Noise Attenuation Level Provided by a Hearing Protector

Many industrial processes generate high levels of noise that can potentially damage human hearing. Although the noise level should ideally be reduced at the source via engineering control, it is often too costly or otherwise impractical. Instead, personal protective equipment (PPE) is used, and often required, to reduce the noise exposure of an individual worker to an acceptable level, and to allow prolonged exposure without resulting in hearing damage.

PPE for noise exposure usually comprise various types of hearing protectors, primarily in the form of earmuffs that completely cover the entire ear, including the outer ear, of the user, or earplugs, which are inserted into the ear canal. Other types of hearing protectors include semi-aural hearing protectors that cover only the entrance to the ear canal.

The level of noise attenuation, or noise reduction, provided by hearing protective devices varies widely across individuals for many reasons, including proper selection and sizing of the device, individual dexterity, and level of skill and training in fitting the devices. Individual physiological characteristics are also important to the degree of protection provided by hearing protective devices.

Evidently, it is important to know the actual magnitude of noise attenuation provided to the individual end-user. For example, this value can be used to determine if the individual is being sufficiently protected simply by measuring the ambient noise and then calculating personal exposure. Individual fit-testing of hearing protectors on end-users has recently become popular.

Two fundamental types of hearing protector fit-testing systems are currently on the market. One is based on human responses to an audiometric hearing evaluation at one or several test frequencies. These are often referred to as Real Ear Attenuation Test (REAT) devices. Briefly, two hearing tests are administered to the test subject, one with hearing protectors in place and one without the hearing protectors. The noise attenuation is then calculated as the difference in hearing threshold at each test frequency. This type of test is embodied in several commercially available systems, including the Michael & Associates, Inc., FitCheck™ system. Disadvantages of this type of device include the inherent variability of human responses to audiometric stimuli, and the time required to implement the test, as one multi-frequency test can take up to 15 minutes to complete. And, some individuals have difficulty recognizing test stimuli due to tinnitus (ears ringing) or due to other cognitive difficulties. This method generally is performed with the test subject wearing headphones and therefore it is not applicable to earmuffs.

The second type of hearing protector fit-testing system is based on objective microphone measurements and is referred to as Field Microphone in Real Ear, or F-MIRE. This type of system is embodied by the Aearo/3M EARFit device, which makes measurements using two microphones mounted on special probed insert-type hearing protectors. Briefly, the test subject wears the special hearing protectors with microphones mounted interior and exterior to the earplug. The special probed earplugs are representative of the plug that the subject wears on a daily basis. The test procedure involves exposing the subject is to a safe level of broadband noise. Both interior and exterior microphones sample and measure the noise. The attenuation in each frequency band is calculated as the difference in magnitude between the two microphone measurements with correction factors applied to account for the different measurement positions. This type of measurement utilizing two microphones is called a noise reduction test. Advantages of the F-MIRE test are that the test procedure does not depend on human responses, and therefore is not subject to the inherent variability, and that the measurement procedure is fast, requiring only seconds to complete the measurement part of the test.

The probed hearing protectors, however, are special versions of Aearo/3M earplugs only, therefore this method is not applicable to other brands of hearing protectors, nor is it applicable to earmuffs.

The method and apparatus according to the present invention overcomes the shortcomings of both types of hearing protector fit-testing systems described above. According to the present invention, a foam-mounted microphone, placed inside a user's ear canal, can be used with both ear-muff type, or earplug type, hearing protectors, without being limited to a particular model or manufacturer's product. For earplug type hearing protectors, the foam-mounted microphone would be placed deep or semi-deep inside the user's ear canal. For muff-type hearing protectors, a shallow mounting is sufficient.

In accordance to one embodiment of the present invention, an F-MIRE field testing method, performed with one microphone mounted in each ear of the test subject using microphones encased in foam ear dams, is provided. In this method, the ear dam-mounted microphones are located in the ear canals, and the subject is exposed to test stimuli (of the same sound level) twice, once with the ears open and once with the ears occluded with the hearing protector. The attenuation at each frequency band is calculated as the arithmetic difference between the two measurements. This is referred to as an insertion loss measurement.

According to one embodiment of the method of the present invention, the analyzer measures noise directed to a test subject in two short bursts. First, with the sub-miniature microphones mounted in both ear canals without hearing protectors in place, the test subject will be exposed to about 80 dBA of broadband noise (covering all frequencies from 50-10000 Hz) at zero degree incidence (directed toward the face from a distance of about 1 m), which is a safe noise exposure level. Other angles of incidence are also acceptable. The test subject will then don hearing protectors on both ears, either earplugs or earmuffs. The thin FFC extending under the hearing protectors has a negligible leakage effect on overall hearing protector attenuation. The subject will then be exposed to same noise burst and a second measurement will be made on the spectrum analyzer.

In case the resultant measurement is compromised by the noise floor of the instrumentation, the sound level of the test stimulus can be increased. For example, a fixed attenuator of approximately 20 dB, often used in the equipment that generates the test stimulus, can be removed from the noise source circuit, which will allow the subject to be exposed to a third (higher level) noise burst. The subject will be safe in this situation as the second measurement has confirmed that the hearing protectors are providing sufficient attenuation so that all exposures experienced by the subject are less than 80 dBA. If the second noise burst measurement is above the noise floor of the instrumentation, the third exposure is not necessary.

The method and apparatus of the present invention are advantageous in that it is fast, safe to the test subject, and applicable to all types of hearing protector devices.

In another embodiment, a multi-channel analyzer may be used, wherein more than one subject can be tested simultaneously, which will further increase the speed of the testing. Multi-channel analyzers are commercially and readily available to those skilled in the art, for example the National Instruments Model 779688-01 digital signal analyzer can be used in a 4-channel configuration to test both ears of two test subjects simultaneously, or an 8-channel device can be used to test both ears of four test subjects simultaneously, and so on. Only one sound source is required for these multi-subject scenarios.

### Sound Delivery System

Conventionally, personal delivery of sound is either via a headphone or an earphone. Headphones can be designed in either a supra aural or circum aural configuration. In the case of the former, the headphone rests on top of the ear with the interface to the wearer typically being soft open-cell foam. In the case of the latter, the ear cup completely encloses the ear with the human-headphone interface typically being a foam based vinyl-type ear pad.

Headphones are typically large and comprise a headband that can either be worn on top of the head or behind the neck. Headphones can be clumsy, bulky and uncomfortable, especially for those who occupy confined spaces. In warm climates, headphones are often rejected since they cause perspiration and are generally considered to be ‘hot’.

An earphone is also called an “ear bud” which is placed directly in or adjacent to the auditory canal. Known earphones generally comprise one or two small audio transducers that are placed directly in or adjacent to the auditory canal. Earphones are used widely with hands-free cellular phone kits and portable audio devices such as Ipod and DVD players. Earphones can be difficult to locate within the ear, leading to user discomfort, and in some cases poor performance for the user. Incorrect fit can also lead to earphones falling from the user's ear.

### Sound Delivery in Noisy Environment

In a preferred embodiment, the foam-mounted sub-miniature speaker of the present invention is coupled with a conventional hearing protection device, either an earmuff-type, or an earplug-type, for communication with a user who works in a high-noise environment. The hearing protection is not compromised in any way, despite the placement of the speaker in the user's ear canal and the presence of the FFC. The combination of the speaker and hearing protector delivers superior sound to the user without the interference of the ambient noise. For example, a sub-miniature speaker can be encased in foam and fitted in the ear canal. The foam contacts the ear canal walls and prevents it from moving and a thin FFC connects the speaker to a communication radio. A conventional earplug and/or earmuff is fitted over the FFC, occluding the ear and minimizing the adverse effects of the ambient noise. Communication or warning signals are received by the radio and transmitted to the ear canal speaker, and effective communication is accomplished in high ambient noise levels. The wearer maximizes comfort by wearing whatever hearing protective device he or she is most accustomed to wearing.

### Sound Delivery With High Situational Awareness

In another embodiment, the ear canal-mounted miniature speaker is used without a hearing protector. In this situation, the user can receive clear communication through the speaker and also be uncompromisingly aware of his or her environment since the non-occluding foam material will have a negligible effect on hearing ambient sounds. This is suitable if the system is used e.g. in a relatively quiet environment. Also, it is suitable in police and military situations where situational awareness is critical.

Another embodiment of the ear canal mounted miniature speaker involves the use of a surveillance earphone. The ear canal mounted transducer is practically invisible from others making possible the secret and private reception of radio communications. Again this is valuable for police and military applications.

Although the invention herein has been described with reference to particular embodiment, it is to be understood that these embodiments merely illustrate the principles and applications of the present invention. It is to be understood that numerous modifications may be devised without departing from the spirit and scope of the present invention as defined by the appended claims.

What is claimed is:

1. An ear-canal mounted sound transducer system, comprising a miniature sound transducer mounted in a soft, acoustically transparent material, suitable for shallow, semi-deep or deep placement inside an ear canal of a user, wherein the sound transducer is attached to a flat flexible cable.

2. The sound transducer system of claim 1, wherein the flat flexible cable is further attached to a wire.

3. The sound transducer system of claim 1, wherein the acoustically transparent material is configured to be easily removable by the user for replacement.

4. The sound transducer system of claim 1, wherein the sound transducer is a microphone.

5. The sound transducer system of claim 1, wherein the sound transducer is a loudspeaker.

6. The ear-canal mounted sound transducer system according to claim 1, wherein the acoustically transparent material is an open-cell foam that contacts the ear canal walls and holds the transducer in place.

7. The sound transducer system of claim 1, wherein the FFC is replaced by wireless transmission.

8. A sound insulated sound transduction system, comprising a hearing protective device, and an ear-canal mounted sound transducer system, wherein the ear-canal mounted sound transducer system comprises a miniature sound transducer mounted in a soft, acoustically transparent material, suitable for placement inside an ear canal of a user, wherein the sound transducer is attached to a flat flexible cable.

9. The sound insulated sound transduction system according to claim 8, wherein the hearing protective device is an ear muff.

10. The sound insulated sound transduction system according to claim 8, wherein the hearing protective device is an ear plug.

11. The sound insulated sound transduction system according to claim 8, wherein the flat flexible cable is further attached to a wire.

12. The sound insulated sound transduction system according to claim 8, wherein the acoustically transparent material is configured to be easily removable by the user for replacement.

13. The sound insulated sound transduction system according to claim 8, wherein the acoustically transparent material is an open-cell foam, or other fibrous material or other structure that contacts the ear canal walls and holds the transducer in place.

14. The sound insulated sound transduction system according to claim 8, wherein the sound transducer is a microphone.

15. The sound insulated sound transduction system according to claim 8, wherein the sound transducer is a loudspeaker.

16. The sound insulated sound transduction system according to claim 8, wherein the sound transducer is a combination of both speaker and loudspeaker.

17. The sound insulated sound transduction system according to claim 8, wherein the FFC is replaced by wireless transmission.

18. A method for measuring the level of noise attenuation of a hearing protecting device worn by a user, the method comprising
1) placing in the ear canal of the user an ear dam-mounted microphone according to claim 4,
2) administering to the user a first test sound stimulus and a second test sound stimulus, wherein the first test stimulus is administered with the ear open and the second test stimulus is administered with the ears occluded with the hearing protective device;
3) measuring the sound level detected at the microphone to obtain a first measurement without the hearing protective device on, and a second measurements with the hearing protective device on, and
4) determining the level of sound attenuation of the hearing protective device.

19. The method according to claim 18, wherein the energy level of the first ant the second test sound stimuli are the same and the level of sound attenuation is determined by calculating the arithmetic difference between the two measurements.

20. The method according to claim 18, wherein the energy level of the second test stimulus is higher than the first test stimulus by a fixed amount X, and the level of sound attenuation is the sum of the arithmetic difference between the two measurements plus X.

21. The method according to claim 18, further comprising administering to the user a third test stimulus which is higher in energy level than the first test stimulus by an amount X, and obtaining a third measurement, wherein the level of sound attenuation is the sum of the arithmetic difference between the first and third measurements, plus X.

22. The method according to claim 18, wherein a plurality of users are tested simultaneously.

23. The method according to claim 18, wherein the hearing protection device is an ear muff.

24. The method according to claim 18, wherein the hearing protection device is an ear plug.
